(19) United States
(12) Patent Application Publication
(10) Pub. No.: US 2025/0232879 A1
(43) Pub. Date: Jul. 17, 2025

(54) METHOD OF PREDICTING ADVERSE REACTIONS TO DRUGS BASED ON TEMPORAL CORRELATION ANALYSIS

(71) Applicant: Univ. Electronic Science and Technology of China, Chengdu (CN)

(72) Inventors: Yongguo LIU, Chengdu (CN); Jiajing ZHU, Chengdu (CN); Yun ZHANG, Chengdu (CN); Qiaoqin LI, Chengdu (CN); Zhi CHEN, Chengdu (CN)

(21) Appl. No.: 18/795,252

(22) Filed: Aug. 6, 2024

(30) Foreign Application Priority Data
Jan. 16, 2024 (CN) 202410059657.9

(51) Int. Cl. G16H 50/30; G16H 10/60; G16H 20/10

(52) U.S. Cl. CPC G16H 50/30; G16H 10/60; G16H 20/10

(57) ABSTRACT

A method for predicting adverse drug reactions based on temporal association analysis, in the field of biomedicine, is disclosed. The method analyzes temporal associations and/or relationships between symptoms, drugs, and adverse reactions at different times, based on temporal sequences of patients' symptoms, the drugs, and the adverse reactions after illness, revealing the association between adverse reactions at the current time and the symptoms, drugs, and adverse reactions at a previous time, and combines the potential relationships between the symptoms, multi-attribute information of the drugs, and the adverse reactions to construct a predictive model for adverse drug reactions based on temporal association analysis, revealing the temporal relationship of adverse drug reactions. The method promotes drug safety, provides data support for the construction of a warning system for adverse drug reactions, and enables prevention and/or treatment of the adverse drug reactions, as well as prevention of potentially ineffective drug treatments.

### RELATED APPLICATIONS

The present application claims priority to Chinese Pat. Appl. No. 202410059657.9, filed Jan. 16, 2024, incorporated by reference herein in its entirety.

### TECHNICAL FIELD

The present invention relates to the field of predicting adverse drug reactions, more specifically, to a method for predicting adverse drug reactions based on temporal correlation analysis.

### BACKGROUND

Adverse drug reactions refer to harmful or unintended reactions that occur when drugs are used for prevention, diagnosis, and treatment of diseases, or physiological regulation, and are unrelated to the intended therapeutic effect. Causes of adverse reactions include prolonged drug use, medication errors, errors in drug administration techniques, and inadequate patient monitoring after medication. For example, beta blockers, a class of antihypertensive drugs, may cause adverse reactions such as hypotension, bradycardia, dizziness, and fatigue. Adverse drug reactions have become an important factor in delaying disease treatment, which can worsen patient conditions and affect morbidity and mortality rates. They are gradually receiving attention from relevant medical and health institutions and have become a key focus of current research in the medical and health fields. To address this issue, pharmaceutical companies invest substantial funds in pre-market clinical trials for adverse reactions during drug development, aiming to reduce the risk of adverse reactions and enhance drug safety. Since adverse drug reactions are highly correlated with the biochemical properties of drugs themselves, such as molecular structure, side effects, targets, enzymes, and pathways, and because different patients (such as those with chronic underlying diseases, children, and the elderly) have varying abilities to absorb and metabolize the same drugs, clinical adverse reaction trials cannot provide extensive and in-depth analysis and research on various drug usage situations and human conditions. On the other hand, due to the large number of drugs available, high costs, and long duration of clinical trials, it is impractical to conduct large-scale identification and prediction of adverse reactions when multiple drugs are used in combination. Therefore, there is a need for effective methods for predicting adverse drug reactions. This is crucial for reducing the cost of new drug development, improving the efficiency of drug development, reducing the occurrence of adverse drug reaction events, and enhancing the level of clinical drug monitoring and early warning.

The current research on predicting adverse drug reactions can be broadly categorized into two types: knowledge-based methods and drug attribute-based methods.

The method of predicting adverse drug reactions based on knowledge typically involves utilizing social media knowledge bases, medical literature, electronic medical record databases, and drug isomer databases. Typical research methods include signal detection and text mining. McMaster et al. proposed a method based on deep natural language processing algorithms, which first pre-trained a large-scale language model, DeBERT, on 1.1 million unlabeled clinical adverse reaction texts, and then fine-tuned the model using 861 labeled adverse reaction texts to predict drug adverse reactions in hospital discharge summaries [C. McMaster, J. Chan, D. F. Liew, et al. Developing a deep learning natural language processing algorithm for automated reporting of adverse drug reactions. Journal of Biomedical Informatics, 2023, 137: 1-10].

The method of predicting adverse drug reactions based on drug attributes involves extracting drug attribute information from drug databases (DrugBank, KEGG, SIDER, PubChem, etc.), and then designing learning models to explore the implicit relationship between drug attributes and adverse drug reactions, thus achieving the prediction of adverse drug reactions. Common research methods include multi-task learning, feature selection, and graph convolutional neural networks, among others. Munoz et al. constructed a drug multi-attribute knowledge graph based on information such as drug type, molecular structure, genes, pathways, and side effects. They transformed adverse reaction prediction into a multi-label ranking problem, established machine learning models including decision trees, random forests, k-nearest neighbor clustering, multi-layer perceptrons, and linear regression, to predict potential adverse drug reactions [E. Muñoz, V. Nováček, P. Y. Vandenbussche. Facilitating prediction of adverse drug reactions by using knowledge graphs and multi-label learning models. Briefings in Bioinformatics, 2019, 20(1): 190-202].

The above methods lay a solid foundation for predicting adverse drug reactions between drugs. However, there are shortcomings: existing models for predicting adverse drug reactions only analyze whether adverse drug reactions occur after patients take specific drugs at the current time. Adverse drug reactions at the current time are also influenced by symptoms, drugs, and adverse reactions in previous time steps. Existing work lacks consideration of the temporal regularities in causing adverse drug reactions. For example, after a person is infected with the novel coronavirus, they typically go through five stages: an “incubation period,” “early infection,” a “progressive stage,” an “advanced stage,” and a “recovery period.” Symptoms evolve with the stages of the disease and have temporal characteristics. The drugs taken by the patients having the symptoms also have temporal characteristics, and the resulting adverse drug reactions follow temporal patterns. Taking a patient's symptom time series “<fever>, <fever>/<sore throat>/<cough>, <cough>/<runny nose>, <runny nose>/<nausea>/<vomiting>, and <nausea>/<vomiting>” as an example (e.g., of one or more symptoms of COVID-19 in successive stages of the disease), the recommended drug time series would be “<acetaminophen>, <chlorpheniramine>/<bromhexine>, <bromhexine>/<loratadine>, <loratadine>/<promethazine>, and <promethazine>.” Due to the temporal correlations between symptoms, drugs, and adverse reactions at different time steps, the resulting adverse reactions also exhibit temporal patterns. Additionally, adverse drug reactions are not only highly correlated with the patient's current symptoms and drug information but also influenced by the symptoms, drugs, and adverse reactions in previous time steps. Therefore, exploring the temporal regularities of adverse drug reactions by analyzing the temporal correlations of symptoms, drugs, and adverse reactions at different time steps is an issue that needs to be addressed.

### SUMMARY OF THE INVENTION

The present invention addresses the problem of existing adverse drug reaction prediction models, which only analyze whether adverse drug reactions occur after patients take specific drugs at the current time, and there is insufficient consideration of the temporal regularities in causing adverse drug reactions in existing work. It provides a method for predicting adverse drug reactions based on temporal correlation analysis.

The invention utilizes time-series information on patient symptoms, drugs, and adverse reactions after the patient becomes ill. It constructs transition probabilities of symptoms, drugs, and adverse reactions at different time points, time stamps or time steps to analyze the temporal correlations among symptoms, drugs, and adverse reactions at different time points, time stamps or time steps (hereinafter, each of the terms “time point,” “time stamp” and “time step” may include the others, and any or all of the terms may be represented by the term “time”). Regarding the symptoms and drug information at the current time, combined with the multi-attribute information (e.g., multi-attribute data) of drugs, it explores the potential relationships between symptom(s), multi-attribute information of drugs, and the adverse reactions. By combining the temporal correlations between different time points or time steps and the potential relationships between symptoms, drugs, and adverse reactions at the current time or time step, it constructs a prediction model for adverse drug reactions based on temporal correlation analysis, revealing the temporal regularities and/or relationships of adverse drug reactions (e.g., with current or earlier symptoms and/or currently- or previously-administered drugs). Therefore, the technical solution of the present invention is a method for predicting adverse drug reactions based on temporal correlation analysis, comprising:

Collecting time-series (temporal) data on one or more symptoms, one or more drugs, and one or more adverse reactions of one or more patients with one or more specific diseases, collecting multi-attribute information (e.g., data) of drugs including molecular structure, targets, pathways, side effects, and phenotypes, denoting a temporal sequence of the symptom(s) as <s₀, s₁, s₂, . . . , sᵢ, . . . , sₙ>, where sᵢ represents at least one of the symptoms of the patient at time tᵢ (e.g., sᵢ∈<s₀, s₁, s₂, s₃, . . . , sₙ₋₁, sₙ>), denoting a temporal sequence of the drug(s) as <m₀, m₁, m₂, . . . , mᵢ, . . . , mₙ>, where mᵢ represents at least one of the drug(s) taken by or administered to the patient at time tᵢ (e.g., mᵢ∈<m₀, m₁, m₂, . . . , mₙ₋₁, mₙ>), and denoting a temporal sequence of the adverse reaction(s) as <r₀, r₁, r₂, . . . , rᵢ, . . . , rₙ> (e.g., rᵢ∈<r₀, r₁, r₂, . . . , rₙ₋₁, rₙ>), where rᵢ represents at least one of the adverse reaction(s) experienced by the patient after taking or being administered the drug(s) at time tᵢ, i∈{0, 1, 2, . . . , n}, n represents the number of time points or time stamps, and collecting attribute feature information (e.g., data) of the drug(s), where each of the drug(s) may have two or more attributes (e.g., the multi-attribute information comprises two or more attributes selected from molecular structure, targets, pathways, side effects, and phenotypes), each of the attributes may have one or more features, the feature information of the v th attribute for the drug(s) is represented as X^{v}∈j^{N×L}^{ᵥ}, N represents a number of the drug(s), Lᵥ represents the number of feature dimensions of the v th attribute, v=1, 2, . . . , V; and V represents the total number of the attributes;

Calculating the temporal correlation among the symptom(s), the drug(s), and the adverse reaction(s) at different time points by calculating a probability p(sᵢ|sᵢ₋₁, sᵢ₋₂, . . . , s₀) of the symptom(s) sᵢ occurring at a given time tᵢ using the following formula:

$$p ( {s}_{i} \vert {s}_{i - 1} , {s}_{i - 2} , … , {s}_{0} ) = \frac{p ( {s}_{i} , {s}_{i - 1} , {s}_{i - 2} , … , {s}_{0} )}{p ( {s}_{i - 1} \vert {s}_{i - 2} , … , {s}_{0} ) p ( {s}_{i - 2} , … , {s}_{0} )}$$

where p(sᵢ|sᵢ₋₁, sᵢ₋₂, . . . , s₀) represents the probability of a first set or subset of the symptom(s) sᵢ occurring at the time tᵢ given the temporal sequence of the symptom(s) <s₀, s₁, . . . , sᵢ₋₁> at a first set or subset of the times or time points t₀˜tᵢ₋₁, and p(sᵢ, sᵢ₋₁, sᵢ₋₂, . . . , s₀) represents the probability of a second set or subset of the symptom(s) <s₀, s₁, . . . , sᵢ> occurring at a second set or subset of the times or time points t₀˜tᵢ, calculating a probability p(rᵢ|sᵢ, mᵢ) of the adverse reaction(s) rᵢ occurring at the given time tᵢ using the following formula:

$$p ( {r}_{i} \vert {s}_{i} , {m}_{i} ) = p ( {s}_{i} \vert {s}_{i - 1} , {s}_{i - 2} , … , {s}_{0} ) \times p ( {m}_{i} \vert {s}_{i} ) \times h ( {s}_{i} , {m}_{i} ) \times {\prod }_{j = 0}^{i - 1} {w}_{i j} [ p ( {r}_{j} \vert {s}_{j} , {m}_{j} ) p ( {m}_{j} , {s}_{j} ) ]$$

where p(mᵢ|sᵢ) represents the probability of the drug(s) mᵢ being taken or administered based on the symptom(s) sᵢ (or the temporal sequence thereof), h(sᵢ, mᵢ) represents a mapping relationship between the symptom(s) sᵢ, the drug(s) mᵢ and the adverse reaction(s) rᵢ at a certain time (e.g., the time tᵢ) p(rⱼ|sⱼ, mⱼ) represents the probability of the adverse reaction(s) occurring based on at least one prior symptom sⱼ and at least one prior drug mⱼ at a prior time tⱼ, p(mⱼ, sⱼ) represents the joint probability of the prior symptom(s) sⱼ occurring and the prior drug(s) mⱼ being taken or administered at the prior time tⱼ, and wᵢⱼ represents a weight of the prior symptom(s) sⱼ and the prior drug(s) mⱼ at the prior time tⱼ on the one adverse drug reaction(s) rᵢ at time tᵢ (e.g., the extent to which the adverse reaction[s] rᵢ at time tᵢ is influenced by the prior symptom[s] sⱼ and the prior drug[s] mⱼ, optionally by an amount that the prior symptom[s] sⱼ and the prior drug[s] mⱼ caused a prior adverse reaction rⱼ), considering the temporal correlation between the time tᵢ and the prior time tⱼ;

Establishing a relationship between the symptom(s), the multi-attribute information of the drug(s), and the adverse reaction(s) by constructing a first similarity matrix Eₘ_{ᵢ}^{v}, for the v-th attribute of the drug(s) mᵢ (e.g., by calculating the similarities between and/or among the features of the v-th attribute of the drug(s) mᵢ that may be administered to or taken by the patient at a prior or successive time point); obtaining information of potential targets related to the symptoms and constructing a second similarity matrix Eₛ_{ᵢ} for the symptom(s) sᵢ based on a similarity between features of the potential targets, where the features of the potential targets are based on the body parts affected by the symptom(s);

Establishing a first interaction relationship Uₘ_{ᵢ}_{, s}_{ᵢ}^{v} from the first similarity matrix Eₘ_{ᵢ}^{v} and the second similarity matrix Eₛ_{ᵢ} as follows:

$${U}_{{m}_{i} , {s}_{i}}^{v} = {\sum }_{a , b} {( {E}_{{m}_{i}}^{v} )}_{a}^{T} \cdot {( {E}_{{s}_{i}} )}_{b} .$$

where (Eₘ_{ᵢ}^{v})_{a·}^{T}, represents the transpose of an a th row of the first similarity matrix Eₘ_{ᵢ}^{v}, (Eₛ_{ᵢ})_{b·} represents a b th row of the second similarity matrix Eₛ_{ᵢ}; and Uₘ_{ᵢ}_{, s}_{ᵢ}^{v} is an interaction relationship (e.g., the first interaction relationship) between the v-th attribute of the drug(s) mᵢ and the potential target(s) of the symptom(s) sᵢ;

Establishing a second interaction relationship between the attributes of the drug(s) mᵢ and the symptom(s) s according to:

$${K}_{{m}_{i} , {s}_{i}} = {\sum }_{v} {U}_{{m}_{i} , {s}_{i}}^{v} \qquad ( 5 )$$

and constructing an objective function mapping of the symptom(s) and the attributes of the drug(s) to the adverse reaction(s) according to:

$$min {\sum }_{i = 0}^{n} {‖ {r}_{i} - f ( {K}_{{m}_{i} , {s}_{i}} ) ‖}_{2}^{2} + Ω ( f ) \qquad ( 6 )$$

where ƒ represents a mapping function between the drug(s), the symptom(s) and the adverse reaction(s), Ω(ƒ) represents a regularization function of or for implicit variables in the function ƒ, the mapping function ƒ integrates a second mapping relationship between (i) the second interaction relationship Kₘ_{ᵢ}_{, s}_{ᵢ} of the drug(s) mᵢ and the symptom(s) s₁ and (ii) the adverse reaction(s) rᵢ at the times or time points, and optimizing the mapping function ƒ to update one or more model parameters using a stochastic gradient descent method;

Constructing a drug adverse reaction prediction model (e.g., based on the temporal correlation analysis) as follows:

$$p ( {r}_{i} \vert {s}_{i} , {m}_{i} ) = p ( {s}_{i} \vert {s}_{i - 1} , {s}_{i - 2} , … , {s}_{0} ) \times p ( {m}_{i} \vert {s}_{i} ) \times f ( {K}_{{m}_{i} , {s}_{i}} ) \times {{\prod }_{j = 0}}^{i - 1} {w}_{ij} [ p ( {r}_{j} \vert {s}_{j} , {m}_{j} ) p ( {m}_{j} , {s}_{j} ) ]$$

where p(rᵢ|sᵢ, mᵢ) is a probability of the patient(s) having the adverse reaction(s) rᵢ at the time tᵢ; and predicting a drug adverse reaction of the patient(s) at a next time point based on the drug adverse reaction prediction model.

Furthermore, calculating the probability p(rᵢ|sᵢ, mᵢ) of the adverse reaction rᵢ at the time tᵢ can also include: ignoring or disregarding the time points distant from the time tᵢ on the adverse reaction(s) at the time tᵢ, and considering only a temporal correlation of adjacent time points tᵢ−tⱼ≤τ. That is, the adverse reaction(s) rᵢ at the time tᵢ is influenced only by the symptom(s), the drug(s), and the adverse reaction(s) at the times t_{i-τ}˜tᵢ₋₁ as follows:

$$p ( {r}_{i} \vert {s}_{i} , {m}_{i} ) = p ( {s}_{i} \vert {s}_{i - 1} , {s}_{i - 2} , … , {s}_{i - τ} ) \times p ( {m}_{i} \vert {s}_{i} ) \times h ( {s}_{i} , {m}_{i} ) \times {\prod }_{j = i - τ}^{i - 1} {w}_{i j} [ p ( {r}_{j} \vert {s}_{j} , {m}_{j} ) p ( {m}_{j} , {s}_{j} ) ]$$

The probability p(r₀|s₀, m₀) of an initial adverse drug reaction or set of adverse drug reactions r₀ occurring at an initial time t₀ may be:

$$p ( {r}_{0} \vert {s}_{0} , {m}_{0} ) = p ( {s}_{0} ) \times p ( {m}_{0} \vert {s}_{0} ) \times h ( {s}_{0} , {m}_{0} )$$

The drug adverse reaction prediction model based on the temporal correlation analysis can also be represented as:

$$p ( {r}_{i} \vert {s}_{i} , {m}_{i} ) = p ( {s}_{i} \vert {s}_{i - 1} , {s}_{i - 2} , … , {s}_{i - τ} ) \times p ( {m}_{i} \vert {s}_{i} ) \times f ( {K}_{{m}_{i} , {s}_{i}} ) \times {\prod }_{j = i - τ}^{i - 1} {w}_{ij} [ p ( {r}_{j} \vert {s}_{j} , {m}_{j} ) p ( {m}_{j} , {s}_{j} ) ] .$$

Furthermore, when the specific disease includes COVID-19, the symptom(s) may include fever, sore throat, cough with sputum, runny nose, nausea and/or vomiting, and the temporal sequence of the symptom(s) may be 1. Fever, 2. Fever, sore throat and/or cough with phlegm, 3. Cough with phlegm and/or runny nose, 4. Runny nose, nausea and/or vomiting, and 5. Nausea and/or vomiting.

Also, when the specific disease includes COVID-19, the drug(s) may include Acetaminophen, Hydroxychloroquine, Bromhexine, Chlorpheniramine and/or Promethazine, and the temporal sequence of the drug(s) may be 1. Acetaminophen, 2. Chloroquine, Bromhexine, 3. Bromhexine and/or Chlorpheniramine, 4. Chlorpheniramine and/or Promethazine, and 5. Promethazine.

And when the specific disease includes COVID-19, the adverse reactions may include indigestion, rash, liver and kidney impairment, fatigue, dizziness, gastric pain, diarrhea, constipation, drowsiness, itching, headache, tremor, dry mouth, blurred vision and/or arrhythmia, and the temporal sequence for the adverse reaction(s) may be 1. Indigestion, rash, liver damage and/or kidney damage, 2. Fatigue, dizziness, stomach pain, diarrhea and/or constipation, 3. Dizziness, constipation, giddiness, diarrhea and/or rash, 4. Itching, rash, headache, tremors or shivering, dry mouth and/or constipation, and 5. Blurred vision, constipation, tremors or shivering, and/or irregular heartbeat.

Additionally, when the specific disease includes diabetes, the symptoms may include thirst, polyuria, frequent urination, dizziness, blurred vision, shortness of breath, chest pain, palpitations, fatigue, sweating, seizures, blindness and/or metabolic disorders, and the temporal sequence for the symptom(s) may be 1. Thirst, polyuria, frequent urination and/or dizziness, 2. Thirst, blurred vision and/or shortness of breath, 3. Chest pain, palpitations, fatigue and/or blurred vision, and 4. Chest pain, sweating, seizures, blindness and/or metabolic disorders.

Furthermore, when the specific disease includes diabetes, the drugs may include metformin, glipizide, insulin and/or captopril, and the temporal sequence for the drug(s) may be 1. Metformin, 2. Metformin and/or Glipizide, 3. Insulin and/or Glipizide, and 4. Insulin, Metformin and/or Captopril.

And when the specific disease includes diabetes, the adverse reactions may include diarrhea, indigestion, abdominal pain, vomiting, dizziness, chills, nausea, sweating, anxiety, tachycardia, tremors or shivering, and/or palpitations, and the temporal sequence for the adverse reaction(s) may be 1. Diarrhea, indigestion, abdominal pain and/or vomiting, 2. Dizziness, chills, diarrhea, abdominal pain and/or nausea, 3. Sweating, anxiety, tachycardia, abdominal pain, nausea and/or dizziness, and 4. Sweating, anxiety, tremors, dizziness, abdominal pain, vomiting, palpitations and/or vertigo.

Additionally, when the specific disease includes hypertension, the symptoms include dizziness, headache, chest pain, neck discomfort, swelling of limbs, fatigue, blurred vision, numbness in limbs, palpitations and/or chest tightness, and the temporal sequence of the symptom(s) may be 1. Dizziness, headache, chest pain and/or discomfort in the neck, 2. Headache, discomfort in the neck and/or swelling of limbs, 3. Headache, fatigue and/or blurred vision, and 4. Dizziness, numbness in limbs, palpitations and/or chest tightness.

Furthermore, when the specific disease includes hypertension, the drugs may include enalapril, hydrochlorothiazide, amlodipine, losartan and/or propranolol, and the temporal sequence for the drug(s) may be 1. Enalapril, 2. Enalapril and/or Hydrochlorothiazide, 3. Amlodipine and/or Losartan, and 4. Propranolol.

And when the specific disease includes hypertension, the adverse reactions may include fatigue, cough, indigestion, dry mouth, muscle spasms, nausea, edema, palpitations, muscle pain, atrioventricular blockage, drowsiness, dizziness and/or insomnia, and the temporal sequence for the adverse reaction(s) may be 1. Fatigue, cough, indigestion and/or dry mouth, 2. Fatigue, dry mouth, muscle spasms and/or nausea, 3. Cough, mild edema, nausea, palpitations and/or muscle pain, and 4. Atrioventricular block, somnolence, dizziness and/or insomnia.

Additionally, when the specific disease includes coronary heart disease, the symptoms may include chest pain, back pain, chest tightness, tachycardia, shortness of breath, angina and/or arm pain, and the temporal sequence for the symptom(s) may be 1. Chest pain, back pain and/or chest tightness, 2. Chest tightness and/or tachycardia, 3. Chest pain, shortness of breath and/or angina pectoris, and 4. Chest pain, arm pain and/or shortness of breath.

When the specific disease includes coronary heart disease, the drugs may include nitroglycerin, aspirin, bisoprolol and/or verapamil, and the temporal sequence for the drug(s) may be 1. Nitroglycerin, 2. Nitroglycerin and/or Aspirin, 3. Aspirin and/or Bisoprolol, and 4. Aspirin and/or Verapamil.

And when the specific disease includes coronary heart disease, the adverse reactions may include blurred vision, dry mouth, hypotension, nausea, vomiting, upper abdominal discomfort, gastrointestinal discomfort, fatigue, sweating, dizziness, constipation and/or palpitations, and the temporal sequence for the adverse reaction(s) may be 1. Blurred vision, dry mouth and/or hypotension, 2. Blurred vision, nausea, vomiting/upper abdominal discomfort, 3. Vomiting, upper abdominal discomfort, gastrointestinal discomfort, fatigue and/or sweating, and 4. Upper abdominal discomfort, nausea, dizziness, constipation and/or palpitations.

Furthermore, when the specific disease includes chronic kidney failure, the symptoms may include polyuria, cardiac arrhythmia, anorexia, oliguria, fatigue, vomiting, anorexia, sluggishness, gastrointestinal ulcers and/or hematochezia, and the temporal sequence for the symptom(s) may be 1. Polyuria, cardiac arrhythmia and/or anorexia, 2. Oliguria, fatigue, vomiting and/or anorexia, 3. Oliguria, sluggishness and/or gastrointestinal ulcers, and 4. Anorexia, hematochezia and/or oliguria.

When the specific disease includes chronic kidney failure, the drugs may include benazepril, perindopril, hydrochlorothiazide and/or furosemide, and the temporal sequence for the drug(s) may be 1. Benazepril and/or Perindopril, 2. Benazepril and/or Hydrochlorothiazide, 3. Benazepril and/or Furosemide, and 4. Furosemide.

When the specific disease includes chronic kidney failure, the adverse reactions may include headache, dizziness, nausea, cough, dry mouth, muscle spasms, fatigue, thirst, muscle soreness and/or arrhythmia, and the temporal sequence for the adverse reaction(s) may be 1. Headache, dizziness, nausea and/or cough, 2. Headache, dizziness, dry mouth, muscle spasms and/or fatigue, 3. Dizziness, thirst, fatigue, muscle soreness and/or arrhythmia, and 4. Fatigue, muscle soreness and/or arrhythmia.

Compared to traditional methods of adverse drug reaction prediction, the present invention analyzes the temporal correlation between symptoms, drugs, and adverse reactions based on the time-series information of patients' symptoms, drugs, and adverse reactions. It explores the potential relationship between the symptoms, the multi-attribute data of the drugs and the adverse reactions. The prediction of adverse drug reactions is conducted from two aspects: the influence of symptoms, drugs, and adverse reactions at previous time steps on adverse reactions at the current time step, and the influence of symptoms and drugs at the current time step on adverse reactions at the current (or a future) time step.

### DRAWINGS

FIG. 1 is a flowchart for an exemplary embodiment of the present invention.

FIG. 2 is a schematic diagram of for an exemplary temporal correlation analysis of symptoms, drugs, and adverse reactions at different time points in one or more embodiments of the present invention.

### EXAMPLES

The technical effects of this solution are as follows: Based on the time-series information of symptoms, drugs, and adverse reactions after a patient becomes ill, the present invention analyzes the correlation between symptoms, drugs, and adverse reactions at different time points. By combining the potential relationship(s) between the symptoms, the multi-attribute information or data of the drugs, and the adverse reactions, a drug adverse reaction prediction model based on temporal correlation analysis is constructed. This model analyzes the degree of influence of adverse reactions at different time points, revealing the temporal patterns of drug adverse reactions. This invention is of great significance for reducing drug adverse reactions in patients at different stages of illness, promoting medication safety research, facilitating the treatment of adverse drug reactions, and preventing the occurrence of adverse drug reactions.

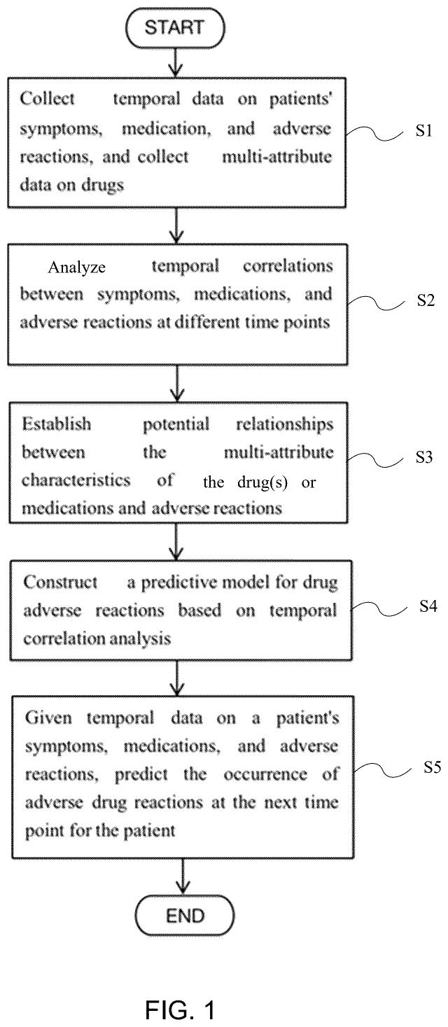

Referring to FIG. 1, in step S1 of a first example, for COVID-19, the Sina Weibo open platform is utilized. The REST API interface (open.weibo.com/wiki/Data_Center) is called to obtain the time-series data of symptoms, drugs, and adverse reactions posted by patients on Weibo, and the data is preprocessed to obtain structured time-series data of symptoms, drugs, and adverse reactions. The molecular structure and target information of drugs are sourced from the DrugBank database, the pathway information of drugs is sourced from the KEGG database, the adverse effects information of drugs is sourced from the SIDER database, and the phenotype information of drugs is sourced from the CTD database. Such attribute information/data may be sourced from other recognized databases as well. This data collection method can cover common symptoms, drugs, and adverse reactions at different stages of specific diseases, as well as multi-attribute data for each drug. Therefore, the data collected by this method is highly reliable.

For the aforementioned multi-attribute information (e.g., data) of drugs, binary vectors are used to represent the attribute features of drugs, where the elements 1 and 0 indicate whether the drug contains the corresponding attribute feature information or not. The sources of drug multi-attribute information and a number of feature dimensions of each attribute are shown in Table 1.

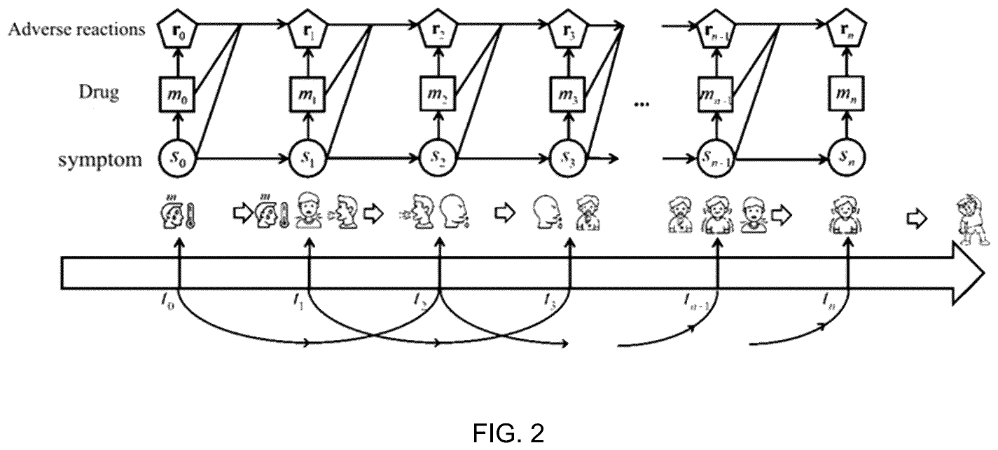

Referring now to FIG. 2, the patient symptom temporal sequence may be denoted as <s₀, s₁, s₂, . . . , sᵢ, . . . , sₙ>, where sᵢ represents one or more symptoms experienced by the patient at time tᵢ (e.g., s₀ represents one or more symptoms experienced by the patient at time t₀, s₁ represents one or more symptoms experienced by the patient at time t₁, sₙ represents one or more symptoms experienced by the patient at time tₙ, etc.). For example, the initial symptom(s) s₀ at the initial time point to in FIG. 2 may be a fever. The symptoms shown in the series in FIG. 2 may be for COVID-19, for example. At the next time point t₁, the symptoms s₁ may include a fever, a sore throat, coughing, or a combination thereof Δt the next time point t₂, the symptoms s₂ may include coughing and/or a runny nose. At the next time point t₃, the symptoms s₃ may include a runny nose and/or vomiting. At a later time point tₙ₋₁, the symptoms sₙ₋₁ may include vomiting, tremors or shivering, a sore throat, or a combination thereof Δt the next (or a final) time point tₙ, the symptoms sₙ may include tremors or shivering. Thus, the symptom(s) sᵢ₊₁ at a next time point tᵢ₊₁ may include one or more of the symptom(s) sᵢ at the previous time point tᵢ. Alternatively, the symptom(s) sᵢ₊₁ at a next time point tᵢ₊₁ may differ from the symptom(s) sᵢ at the previous time point tᵢ, or may include one or more of the symptom(s) sᵢ at the previous time point tᵢ and one or more symptoms different from the symptom(s) sᵢ at the previous time point tᵢ. The term “sᵢ” may thus define a set of one or more symptoms at a time stamp i (e.g., <s₀, s₁, s₂, . . . , sᵢ, . . . , sₙ₋₁, sₙ>), and the term “<s₀, s₁, s₂, . . . , sᵢ, . . . , sₙ₋₁, sₙ>)” may indicate that the set of symptoms of patients at different time stamps are sorted by time stamp. The set of symptoms that any given patient has at the time stamp i in sᵢ may contain one symptom or multiple symptoms. Adjacent or successive time points, such as tᵢ and tᵢ₊₁, may be spaced apart through the temporal sequence (e.g., time series) by the same length of time or different lengths of time. Generally, and consistent with progressions of many common diseases, the symptom(s) sᵢ₊₁ at the next time point tᵢ₊₁ typically include at least one symptom that differs from the symptom(s) sᵢ at the previous time point tᵢ.

The drug temporal sequence is denoted as <m₀, m₁, m₂, . . . , mᵢ, . . . , mₙ>, where mᵢ represents the drug(s) taken by the patient to treat the disease and/or to alleviate symptom(s) sᵢ at time tᵢ (e.g., m₀ represents one or more drugs taken by or administered to the patient at time t₀, m₁ represents one or more drugs taken by or administered to the patient at time t₁, mₙ represents one or more drugs taken by or administered to the patient at time tₙ, etc.). Thus, the term “mᵢ” may thus define a set of one or more drugs administered to or taken by the patient at the time stamp i (e.g., <m₀, m₁, m₂, . . . , mᵢ, . . . , mₙ₋₁, mₙ>), and the term “<m₀, m₁, m₂, . . . , mᵢ, . . . , mₙ₋₁, mₙ>” may indicate that the set of drugs at different time stamps are sorted by time stamp. The set of drugs that any given patient has taken or been given at the time stamp i in mᵢ may contain one drug or multiple drugs. For example, the initial drug(s) m₀ at the initial time point t₀ in FIG. 2 may be paracetamol. At the next time point t₁, the drug(s) m₁ may include chloroquine and/or bromhexine. Thus, the drug(s) mᵢ₊₁ at a next time point tᵢ₊₁ may include one or more drugs different from the drug(s) mᵢ at the previous time point tᵢ. Alternatively, the drug(s) mᵢ₊₁ at the next time point tᵢ₊₁ may be the same as those at the previous time point tᵢ, or may include one or more of the drug(s) mᵢ at the previous time point tᵢ and one or more drugs different from those at the previous time point tᵢ. In many embodiments, only one drug is administered to the patient at one or more of the time points tᵢ, and often, at least one drug mᵢ₊₁ at the next time point tᵢ₊₁ is the same as at least one drug mᵢ at the previous time point tᵢ.

The adverse reaction temporal sequence is denoted as <r₀, r₁, r₂, . . . , rᵢ, . . . , rₙ> where rᵢ represents the adverse reaction(s) experienced by the patient after taking drug(s) mᵢ and experiencing symptom(s) sᵢ at time tᵢ, i is a sequence of non-negative integers (e.g., i∈{0, 1, 2, . . . , n}) and n represents the number of time points. Thus, the term “rᵢ” may thus define a set of one or more adverse reactions of the patient at the time stamp i (e.g., <r₀, r₁, r₂, . . . , rᵢ, . . . , rₙ₋₁, rₙ>), and the term “<r₀, r₁, r₂, . . . , rᵢ, . . . , rₙ₋₁, rₙ>” may indicate that the set of adverse reactions at different time stamps are sorted by time stamp. The set of adverse reactions that any given patient exhibits at the time stamp i in rᵢ may contain one adverse reaction or multiple adverse reactions. For example, r₀ represents one or more adverse reaction(s) experienced by the patient at time t₀, r₁ represents one or more adverse reaction(s) experienced by the patient at time t₁, rₙ represents one or more adverse reaction(s) experienced by the patient at time tₙ, etc. The adverse reaction(s) rᵢ₊₁ at a next time point tᵢ₊₁ may be in response to the drug(s) mᵢ taken by or administered to the patient at the previous time point tᵢ. Referring back to the example shown in FIG. 2, at the next time point t₁, the adverse reaction(s) r₁ (e.g., to the drug(s) m₀ taken by or administered to the patient at the initial time point to) may include fatigue, dizziness, stomach ache, and/or either diarrhea or constipation. Thus, the adverse reaction(s) rᵢ₊₁ at the next time point tᵢ₊₁ may include one or more adverse reactions different from the adverse reaction(s) rᵢ at the previous time point tᵢ. Alternatively, the adverse reaction(s) rᵢ₊₁ at the next time point tᵢ₊₁ may include one or more of the same adverse reactions rᵢ as those at the previous time point tᵢ, or may include a combination of one or more of the same adverse reactions rᵢ at the previous time point tᵢ and one or more adverse reactions different from those at the previous time point tᵢ. In many cases, when there are more than one possible adverse reactions rᵢ, the patient exhibits less than all of the possible adverse reactions rᵢ.

The collected dataset contains a total of N drugs, where N is a positive integer. Based on the drug multi-attribute feature dimension information provided in Table 1, the feature space of the v th attribute of the drugs is represented as X^{v}∈i^{N×L}^{ᵥ} where Lᵥ indicates the feature dimension of the v th attribute, v=1, 2, . . . , V; and V represents the number of attributes. In some embodiments of this invention, V=5, but the invention is not so limited. For example, V can be an integer of 2 to 5, and V can be independent for each drug.

TABLE 1
Information on the Source Databases and Feature
Dimensions of Drug Multi-Attributes

| | | Feature |
|---|---|---|
| Drug Attribute | Database | Dimensions Lᵥ |
| Molecular Structure | DrugBank | 881 |
| Target | DrugBank | 497 |
| Pathway | KEGG | 396 |
| Side effects | SIDER | 3687 |
| Phenotype | CTD | 2193 |

Referring back to FIG. 1, in step S2 of this example, the temporal relationships between symptoms, drugs, and adverse reactions of patients at different time points are analyzed. For example, taking the symptom temporal sequence (e.g., time series) of a patient infected with the novel coronavirus SARS-CoV-2 (e.g., s₁=“fever,” s₂=“fever”/“dry and sore throat”/“coughing with phlegm,” s₃=“coughing with phlegm”/“runny nose,” s₄=“runny nose”/“nausea”/“vomiting,” and s₅=“nausea”/“vomiting”), it is observed that the symptom “fever” occurs at the first and second time points, with the addition of symptoms “dry and sore throat” and “coughing with phlegm” at the second time point. At the third time point, symptoms “fever” and “dry and sore throat” disappear, with the addition of the symptom “runny nose.” This indicates that although the patient's symptoms change over time, there is a correlation between symptoms at different time points, especially between adjacent time points. The correlation between symptoms at different time points may affect the correlation between drugs and adverse reactions between time points. Considering the correlation between symptoms at different time points, the probability of symptom occurrence at a certain time point is influenced by the symptoms at preceding time points. Therefore, the probability of symptom occurrence at a certain time point is represented as:

$$p ( {s}_{i} \vert {s}_{i - 1} , {s}_{i - 2} , … , {s}_{0} ) = \frac{p ( {s}_{i} , {s}_{i - 1} , {s}_{i - 2} , … , {s}_{0} )}{p ( {s}_{i - 1} \vert {s}_{i - 2} , … , {s}_{0} ) p ( {s}_{i - 2} , … , {s}_{0} )} \qquad ( 1 )$$

where p(sᵢ|sᵢ₋₁, sᵢ₋₂, . . . , s₀) represents the probability of the symptom sequence sᵢ occurring at time tᵢ given the temporal sequence of the symptom(s) <s₀, s₁, . . . , sᵢ₋₁> at times or time points t₀˜tᵢ₋₁, which may be based on the occurrence of symptom(s) in the symptom temporal sequence at prior times or time points.

On the other hand, the probability of an adverse drug reaction rᵢ occurring at time tᵢ is highly correlated with the symptom(s) sᵢ of the patient and drug(s) m administered to the patient at time (or time point) tᵢ, and is also influenced by the symptom(s) sⱼ, drug(s) mⱼ, and adverse reaction(s) rⱼ at time (or time point) tⱼ, j≤i, Therefore, based on the probability of symptoms at different times or time points given by Equation (1), the probability p(rᵢ|sᵢ, mᵢ) of adverse reaction(s) rᵢ occurring at time tᵢ is:

$$p ( {r}_{i} \vert {s}_{i} , {m}_{i} ) = p ( {s}_{i} \vert {s}_{i - 1} , {s}_{i - 2} , … , {s}_{0} ) \times p ( {m}_{i} \vert {s}_{i} ) \times h ( {s}_{i} , {m}_{i} ) \times {\prod }_{j = 0}^{i - 1} {w}_{i j} [ p ( {r}_{j} \vert {s}_{j} , {m}_{j} ) p ( {m}_{j} , {s}_{j} ) ] \qquad ( 2 )$$

where p(mᵢ|sᵢ) represents the probability of drug(s) mᵢ administered or given to the patient at time tᵢ upon the occurrence of symptom(s) sᵢ, h(sᵢ, mᵢ) represents the mapping relationship between (a) the symptom(s) sᵢ and the drug(s) mᵢ at time tᵢ and (b) the adverse reaction(s) rᵢ, p(rⱼ|sⱼ, mⱼ) represents the probability of occurrence of the adverse reaction(s) rⱼ when the symptom(s) sⱼ occur in the patient and the drug(s) mⱼ has been administered or given to the patient at time tⱼ, p(mⱼ, sⱼ) represents the joint probability of symptom(s) sⱼ occurring in the patient and the drug(s) mⱼ having been administered or given to the patient at time tⱼ, and weight wᵢⱼ represents the degree of or to which the adverse drug reaction(s) rᵢ at time tᵢ is influenced by the occurrence of the adverse drug reaction(s) rⱼ (which may be triggered by symptom(s) sⱼ) occurring in the patient and drug(s) mⱼ having been administered or given to the patient at time tⱼ. The weight wᵢⱼ can also be regarded as the temporal correlation between time tᵢ and time tⱼ. Intuitively, the greater the time interval tᵢ−tⱼ between time points, the smaller the influence of the symptom(s), drug(s), and adverse reaction(s) at time tⱼ on adverse reaction(s) rᵢ at time tᵢ.

In order to reduce the complexity of Equation (2) and to ignore the influence of distant time points on the adverse reaction(s) at time (or time point) tᵢ, one may consider only the temporal correlation of tᵢ−tⱼ≤τ, where τ is a predetermined unit of time (e.g., 1 day, 6 hours, etc.). In this case, the adverse reaction(s) rᵢ at time (or time point) tᵢ is influenced or affected only by the symptom(s), drug(s), and adverse reaction(s) at times (or time points) t_{i-τ}˜tᵢ₋₁ Therefore, Equation (2) can be further simplified as:

$$p ( {r}_{i} \vert {s}_{i} , {m}_{i} ) = p ( {s}_{i} \vert {s}_{i - 1} , {s}_{i - 2} , … , {s}_{i - τ} ) \times p ( {m}_{i} \vert {s}_{i} ) \times h ( {s}_{i} , {m}_{i} ) \times {\prod }_{j = i - τ}^{i - 1} {w}_{i j} [ p ( {r}_{j} \vert {s}_{j} , {m}_{j} ) p ( {m}_{j} , {s}_{j} ) ] \qquad ( 3 )$$

Specifically, the probability p(r₀|s₀, m₀) of occurrence of adverse drug reaction r₀ at a given time t₀ is expressed as:

$$p ( {r}_{0} \vert {s}_{0} , {m}_{0} ) = p ( {s}_{0} ) \times p ( {m}_{0} \vert {s}_{0} ) \times h ( {s}_{0} , {m}_{0} ) \qquad ( 4 )$$

where m₀, s₀ and r₀ refer respectively to an initial drug or set of drugs, an initial symptom or set of symptoms, and an initial adverse reaction or set of adverse reactions, and p(r₀|s₀, m₀) is the probability of the initial adverse drug reaction(s) r₀ occurring.

In step S3 of FIG. 1, one may explore the potential relationship(s) between the symptom(s), the multi-attribute information (e.g., data) of the drugs, and the adverse reactions. Specifically, one may investigate the method by which the mapping function h(sᵢ, mᵢ) in Equation (2) (e.g., step S2 of FIG. 1) is constructed.

Specifically, regarding the symptom(s) sᵢ at time or time point tᵢ, the adverse reactions triggered by taking drug(s) m; may be explored. Based on the multi-attribute data of the drugs (e.g., molecular structure, target, pathway, side effects, and phenotype), the feature representation of drug(s) m; is obtained from the drug's multi-attribute feature space X, where v=1, 2, . . . , V For the v th attribute feature representation of drug(s) mᵢ, the drug's attribute feature similarity matrix Eₘ_{ᵢ}^{v} may be constructed by calculating the similarity between the features (e.g., among the features of the v-th attribute for drug(s) mᵢ)

$${E}_{{m}_{i}}^{v} = {\sum }_{{d}_{k} ∈ {m}_{i}} {E}_{{d}_{k}}^{v} ,$$

where E_{d}_{ₖ}^{v} is the v-th attribute feature similarity matrix for drug dₖ. If mᵢ contains only one drug, then mᵢ=dₖ. If mᵢ contains more than one drug, drug dₖ is one of the drugs in mᵢ. These two cases can be unified as dₖ∈mᵢ.

Regarding the symptoms sᵢ, the potential target data related to the symptoms may be obtained by analyzing the body parts affected by the symptoms, and optionally, identifying the organ(s), tissue(s), physiological pathway(s), receptor(s), or other physiological structure targeted by the drug(s) mᵢ. Based on the similarity between target features (e.g., the similarity or similarities among the body parts affected by the symptoms), the target feature similarity matrix Eₛ_{ᵢ} of the symptoms sᵢ may be constructed. To investigate the adverse drug reactions triggered by drug(s) mᵢ when treating symptom(s) sᵢ, for any attribute feature similarity matrix Eₘ_{ᵢ}^{v} of drug(s) mᵢ, the interaction relationship Uₘ_{ᵢ}_{, s}_{ᵢ}^{v} between Eₘ_{ᵢ}^{v} and the target feature similarity matrix Eₛ_{ᵢ} of symptom(s) sᵢ can be calculated according to Equation (5) below:

$${U}_{{m}_{i} , {s}_{i}}^{v} = {\sum }_{a , b} {( {E}_{{m}_{i}}^{v} )}_{{a}^{.}}^{T} {( {E}_{{s}_{i}} )}_{{b}^{.}} \qquad ( 5 )$$

where (Eₘ_{ᵢ}^{v})_{a·}^{T}, represents the transpose of the a th row of matrix Eₘ_{ᵢ}^{v}, while (Eₛ_{ᵢ})_{b·} represents the b th row of matrix Eₛ_{ᵢ}. The interaction relationship between the v th attribute feature of drug(s) mᵢ and the target feature(s) of symptom(s) sᵢ may be obtained by summing the results of multiplying any row vector in matrix Eₘ_{ᵢ}^{v} with any row vector in matrix Eₛ_{ᵢ}. Therefore, the interaction relationship between all attributes of drug(s) mᵢ and symptom(s) sᵢ can be expressed as:

$${K}_{{m}_{i} , {s}_{i}} = {\sum }_{v} {U}_{{m}_{i} , {s}_{i}}^{v} \qquad ( 6 )$$

To establish the potential relationship between the interaction Kₘ_{ᵢ}_{, s}_{ᵢ} of drug(s) mᵢ and symptom(s) sᵢ at time tᵢ and the adverse reaction(s) rᵢ, the present invention maps (e.g., constructs a mapping) between Kₘ_{ᵢ}_{, s}_{ᵢ} and rᵢ, achieving the relationship constructed from the symptoms and the multi-attribute data of the drugs to the adverse drug reactions. Therefore, the objective function for mapping symptoms and multi-attribute information of the drugs to the adverse reactions can be written as:

$$min {\sum }_{i = 0}^{n} {‖ {r}_{i} - f ( {K}_{{m}_{i} , {s}_{i}} ) ‖}_{2}^{2} + Ω ( f ) \qquad ( 7 )$$

where ƒ represents the mapping function between (i) the interaction of the drug(s) and the symptom(s) and (ii) the adverse reactions, while Ω(ƒ) represents a regularization function of the implicit variables in the function ƒ. Function ƒ integrates the mapping relationships between (i) the interaction Kₘ_{ᵢ}_{, s}_{ᵢ} of the drug(s) mᵢ with the symptom(s) sᵢ and (ii) the adverse reaction(s) rᵢ at all time points. Utilizing a stochastic gradient descent method, a function ƒ may be optimized to update model parameters.

In step S4 of FIG. 1, a drug adverse reaction prediction model based on temporal correlation analysis is constructed. In one embodiment, the objective function of this prediction model is:

$$p ( {r}_{i} \vert {s}_{i} , {m}_{i} ) = p ( {s}_{i} \vert {s}_{i - 1} , {s}_{i - 2} , … , {s}_{i - τ} ) \times p ( {m}_{i} \vert {s}_{i} ) \times f ( {K}_{{m}_{i} , {s}_{i}} ) \times {\prod }_{j = i - τ}^{i - 1} {w}_{ij} [ p ( {r}_{j} \vert {s}_{j} , {m}_{j} ) p ( {m}_{j} , {s}_{j} ) ] \qquad ( 8 )$$

The aforementioned objective function considers both (i) the influence of the preceding symptom(s), drug(s), and adverse reaction(s) on the current adverse reaction and (ii) the mapping relationship between the current symptom(s), drug(s), and adverse reaction(s). A Markov chain may incorporate sequential data of a patient's symptoms, drug(s), and adverse reaction(s) as observable variables, with the state transition probabilities between time points serving as latent variables. Maximum likelihood estimation may be used to estimate the latent variables by solving the likelihood function (e.g., for the latent variables). Furthermore, variational inference methods may be used to treat the probabilities of a patient's symptoms, drug(s), and adverse reaction(s) as probability distribution functions containing latent parameters (such as mixture Gaussian probability distribution functions, Bernoulli distribution functions, etc.). By minimizing the Kullback-Leibler (KL) divergence between the simple distribution of observable variables and the actual complex distribution, based on the evidence lower bound (ELBO) derived from the probability density of observable variables, one may estimate the latent parameters of the probability distribution functions.

In step S5 of FIG. 1, the objective is to predict the patient's future adverse reaction(s) to the drug(s), given the temporal sequence data of the patient's symptom(s), drug(s), and adverse reaction(s). The future adverse reaction(s) may be for the same drug(s) and/or one or more different drugs. Specifically, the adverse reaction(s) rᵢ at time tᵢ is influenced by the symptom(s), drug(s), and adverse reaction(s) at one or more times or time points t_{i-τ}˜tᵢ₋₁, as well as by the symptom(s) sᵢ and drug(s) mᵢ at the time or time point tᵢ. Given the temporal sequence (e.g., time series) data of the symptoms <s_{i-τ}, s_{i-τ+1}, . . . , sᵢ₋₁>, the drug(s) <m_{i-τ}, m_{i-τ+1}, . . . , mᵢ₋₁>, and adverse reactions <r_{i-τ}, r_{i-τ+1}, . . . , rᵢ₋₁> at the times or time points t_{i-τ}˜tᵢ₋₁, as well as the symptoms sᵢ and drug(s) mᵢ at the time or time point tᵢ, the first step may be to compute the target feature similarity matrix Eₛ_{ᵢ} for symptoms sᵢ and the multi-attribute feature similarity matrix Eₘ_{ᵢ}^{v}, v=1, 2, . . . , V by obtaining the interaction relationship Kₘ_{ᵢ}_{, s}_{ᵢ} between the attributes of the drug(s) m and the symptom(s) sᵢ as inputs for the function ƒ, then ƒ is computed. Next, considering the temporal correlation between the symptom(s), the drug(s), and the adverse reaction(s) at different time points, along with the transition probabilities of adverse reaction(s) between different time points (e.g., from one time point to the next time point), the prediction of adverse reaction(s) rᵢ at time or time point tᵢ may be carried out using Equation (8).

In various embodiments, the present method for predicting drug adverse reactions based on temporal correlation analysis is utilized to explore the temporal patterns of drug adverse reactions, considering the temporal correlations of symptoms, drugs, and adverse reactions at different time points. Some of the predicted results are supported by relevant literature and carry a certain level of credibility. Table 2 presents the temporal sequences of symptoms, drugs, and predicted adverse reactions for patients infected with the novel coronavirus (i.e., COVID-19). Table 3 provides the temporal sequences of symptoms, drugs, and predicted adverse reactions for diabetic patients. Table 4 presents the temporal sequences of symptoms, drugs, and predicted adverse reactions for hypertensive patients. Table 5 provides the temporal sequences of symptoms, drugs, and predicted adverse reactions for patients with coronary heart disease. Table 6 presents the temporal sequences of symptoms, drugs, and predicted adverse reactions for patients with chronic kidney failure.

These tables offer detailed information about the symptoms, drugs, and predicted adverse reactions for different types of patients, aiding in the understanding of the temporal patterns of drug adverse reactions among various patient populations.

TABLE 2
Temporal sequences of symptoms, medications, and predicted adverse
reactions for patients infected with the novel coronavirus

| Time point | 1 | 2 | 3 | 4 | 5 |
|---|---|---|---|---|---|
| Temporal | Fever | Fever, | Coughing | Runny nose, | Nausea |
| sequence of | | sore throat | with | Nausea and/or | and/or |
| symptoms | | and/or cough | phlegm, | vomiting | vomiting |
| | | with phlegm | Runny nose | | |
| Temporal | Paracetamol | Chloroquine | Bromhexine | Chlorpheniramine | Promethazine |
| sequence of | | and/or | and/or | and/or | |
| medications | | Bromhexine | Chlorpheniramine | Promethazine | |
| Temporal | Indigestion, | Fatigue, | Dizziness, | Itching, rash, | Blurred |
| sequence of | rash, | dizziness, | constipation | headache, tremor, | vision, |
| adverse | liver | stomach | or diarrhea, | dry mouth and/or | constipation, |
| reactions | and/or | ache, and/or | headache | constipation | tremor or |
| | kidney | diarrhea or | and/or rash | | shivering, |
| | damage | constipation | | | and/or |
| | | | | | irregular |
| | | | | | heartbeat |

TABLE 3
Temporal sequences of symptoms, medications, and
predicted adverse reactions for diabetic patients

| Time point | 1 | 2 | 3 | 4 |
|---|---|---|---|---|
| Temporal | Thirst, polyuria, | Thirst, blurred | Chest pain, | Chest pain, |
| sequence of | frequent | vision and/or | palpitations, | sweating, |
| symptoms | urination and/or | shortness of | fatigue and/or | seizures, |
| | dizziness | breath | blurred vision | blindness and/or |
| | | | | metabolic |
| | | | | disorder |
| Temporal | Metformin | Metformin | Insulin and/or | Insulin, |
| sequence of | | and/or | Gliclazide | Metformin and/or |
| medications | | Gliclazide | | Captopril |
| Temporal | Diarrhea, | Dizziness, | Sweating, | Sweating, |
| sequence of | Indigestion, | Chills, Diarrhea, | Anxiety, Rapid | Anxiety, Tremors |
| adverse | Abdominal pain | Abdominal pain | heartbeat, | or shivering, |
| reactions | and/or Vomiting | and/or Nausea | Abdominal pain, | Dizziness, |
| | | | Nausea and/or | Abdominal pain, |
| | | | Dizziness | Vomiting, |
| | | | | Palpitations |
| | | | | and/or Vertigo |

TABLE 4
Temporal sequences of symptoms, medications, and predicted
adverse reactions for hypertensive patients

| Time point | 1 | 2 | 3 | 4 |
|---|---|---|---|---|
| Temporal | Dizziness, | Headache, Neck | Headache, | Dizziness, |
| sequence of | Headache, Chest | discomfort | Fatigue and/or | Numbness in |
| symptoms | pain and/or | and/or Swelling | Blurred vision | limbs, |
| | Neck discomfort | in limbs | | Palpitations |
| | | | | and/or Chest |
| | | | | tightness |
| Temporal | Enalapril | Enalapril and/or | Amlodipine | Propranolol |
| sequence of | | Hydro- | and/or Losartan | |
| medications | | chlorothiazide | | |
| Temporal | Fatigue, cough, | Fatigue, dry | Cough, Edema | Atrioventricular |
| sequence of | indigestion | mouth, muscle | (which may be | blockage, |
| adverse | and/or dry | spasms and/or | moderate), | Somnolence, |
| reactions | mouth | nausea | Nausea, | Dizziness and/or |
| | | | Palpitations | Insomnia |
| | | | and/or Muscle | |
| | | | Pain | |

TABLE 5
Temporal sequences of symptoms, medications, and predicted
adverse reactions for patients with coronary heart disease

| Time point | 1 | 2 | 3 | 4 |
|---|---|---|---|---|
| Temporal | Chest pain, Back | Chest tightness | Chest pain, | Chest pain, Arm |
| sequence of | pain and/or | and/or | Shortness of | pain and/or |
| symptoms | Chest tightness | Tachycardia | breath and/or | Shortness of |
| | | | Angina | breath |
| Temporal | Nitroglycerin | Nitroglycerin | Aspirin and/or | Aspirin and/or |
| sequence of | | and/or Aspirin | Bisoprolol | Verapamil |
| medications | | | | |
| Temporal | Blurred vision, | Blurred vision, | Vomiting, Upper | Upper abdominal |
| sequence of | Dry mouth | Nausea, | abdominal | discomfort, |
| adverse | and/or | Vomiting and/or | discomfort, | Nausea, |
| reactions | Hypotension | Upper | Gastrointestinal | Dizziness, |
| | | abdominal | discomfort, | Constipation |
| | | discomfort | Fatigue and/or | and/or |
| | | | Sweating | Palpitations |

TABLE 6
Temporal sequences of symptoms, medications, and predicted
adverse reactions for patients with chronic kidney failure

| Time point | 1 | 2 | 3 | 4 |
|---|---|---|---|---|
| Temporal | Polyuria, | Oliguria, | Oliguria, | Loss of appetite, |
| sequence of | Cardiac | Fatigue, | Drowsiness | Hematochezia |
| symptoms | arrhythmia | Vomiting and/or | and/or | and/or Oliguria |
| | and/or Loss of | Loss of appetite | Gastrointestinal | |
| | appetite | | ulcer | |
| Temporal | Benazepril | Benazepril | Benazepril | Furosemide |
| sequence of | and/or | and/or | and/or | |
| medications | Perindopril | Hydrochlorothiazide | Furosemide | |
| Temporal | Headache, | Headache, | Dizziness, Thirst, | Fatigue, Muscle |
| sequence of | Dizziness, | Dizziness, Dry | Fatigue, Muscle | soreness and/or |
| adverse | Nausea and/or | mouth, Muscle | soreness and/or | Cardiac |
| reactions | Cough | spasms and/or | Cardiac | arrhythmia |
| | | Fatigue | arrhythmia | |

In some embodiments, the method may further comprise determining that the patient(s) will have at least one of the drug adverse reactions at the next time point when the probability p(rᵢ|sᵢ, mᵢ) is equal to or greater than a predetermined threshold. For example, the predetermined threshold may be 0.5 (i.e., there is a 50% chance or higher than the patient will have a drug adverse reaction at the next time point). However, the invention is not so limited, and in practice, the predetermined threshold may be based on the potential or likely severity of the possible adverse reactions. For example, the greater the potential or likely severity of the possible adverse reactions, the lower the predetermined threshold may be. In such cases, the potential or likely severity of the possible adverse reactions may be assigned a value (e.g., on a scale of 0-5 or 1-10), then the predetermined threshold may be 0.5−x or 1−y, where x is the potential or likely severity value on a scale of 0-5, and y is the potential or likely severity value on a scale of 1-10. The values x and y may represent the value for the most severe drug adverse reaction, or it may represent a weighted and/or summed value for all of the drug adverse reactions. In such examples, the predetermined threshold may be in the range of 0.1-0.5, although the invention is not so limited. Alternatively, the predetermined threshold may be independently determined for each of the drugs or each of the adverse reactions.

The invention also concerns a method of preventing one or more adverse reactions to one or more drugs, comprising predicting the adverse drug reaction(s) at the next time point according to the present method, and withholding at least one of the one or more drugs from the one or more patients when the probability p(rᵢ|sᵢ, mᵢ) is equal to or greater than a predetermined threshold, where the predetermined threshold is as discussed above. For example, when more than one drug is taken by or administered to the patient at time tᵢ, all of the drugs may be withheld from the patient at later time points when the probability p(rᵢ|sᵢ, mᵢ) is equal to or greater than the predetermined threshold. Alternatively, when the probability p(rᵢ|sᵢ, mᵢ) is equal to or greater than the predetermined threshold for the combination of drugs, the probability p(rᵢ|sᵢ, mᵢ) may be determined separately for each drug taken by or administered to the patient at time tᵢ, and only the drug having the lowest probability p(rᵢ|sᵢ, mᵢ) (or, when more than two drugs are taken by or administered to the patient at time tᵢ, the subset of drugs having the lowest probabilities p(rᵢ|sᵢ, mᵢ)) may be taken by or administered to the patient at time tᵢ₊₁, as long as that probability p(rᵢ|sᵢ, mᵢ) is also below the predetermined threshold (e.g., for the drug[s] and/or adverse reaction[s]).

In a further aspect, the invention also concerns a method of treating one or more adverse reactions to one or more drugs, comprising administering the drug(s) to the patient(s) after the patient(s) present or exhibit the symptom(s), predicting the adverse drug reaction(s) at the next time point according to the present method, and prior to the patient(s) having or exhibiting the adverse drug reaction(s) at the next time point, either: (i) withholding at least one of the drugs from the patient(s), or (ii) treating the predicted adverse drug reaction(s) (i.e., the one or more adverse drug reactions predicted at the next time point according to the present method) in the patient(s). Typically, this method comprises treating the predicted adverse drug reaction(s) in the patient(s) prior to the patient(s) having or exhibiting the adverse drug reaction(s) at the next time point.

For example, when the predicted adverse drug reactions include nausea and/or vomiting, the predicted adverse drug reaction(s) may be treated by administering an antiemetic drug to the patient(s). Similarly, when the predicted adverse drug reactions include constipation, the predicted adverse drug reaction(s) may be treated by administering a laxative to the patient(s). In addition, when the predicted adverse drug reactions include indigestion, stomach ache, abdominal discomfort and/or abdominal pain, the predicted adverse drug reaction(s) may be treated by administering a digestive aid to the patient(s). Alternatively, when the predicted adverse drug reactions include fatigue, muscle soreness, muscle spasms or muscle atrophy, the predicted adverse drug reaction(s) may be treated by appropriate exercise or physical therapy. It is within the level of skill in the art of medicine (e.g., internal medicine) to determine an appropriate treatment regimen for the predicted adverse drug reactions.

What is claimed:

1. A method for predicting an adverse drug reaction, comprising:
collecting temporal data on one or more symptoms, one or more drugs, and one or more adverse reactions of one or more patients with one or more specific diseases, wherein the one or more patients have the one or more symptoms, are administered the one or more drugs after presenting or having the one or more symptoms, and have the one or more adverse reactions after being administered the one or more drugs, by:
collecting multi-attribute data of the one or more drugs, including molecular structure, targets, pathways, side effects, and phenotypes;
denoting a temporal sequence of the one or more symptoms as <s₀, s₁, s₂, . . . , sᵢ, . . . , sₙ>, where sᵢ represents at least one of the one or more symptoms at a time tᵢ;
denoting a temporal sequence of the one or more drugs as <m₀, m₁, m₂, . . . , mᵢ, . . . , mₙ>, where mᵢ represents at least one of the one or more drugs administered to the patient at the time tᵢ;
denoting a temporal sequence of the one or more adverse reactions as <r₀, r₁, r₂, . . . , rᵢ, . . . , rₙ>, where rᵢ represents at least one of the one or more adverse reactions of the patient after taking or being administered the at least one drug at the time tᵢ, i∈{0, 1, 2, . . . , n}, and n represents a number of time points or time stamps; and
collecting attribute feature information for the one or more drugs, wherein features of a v-th attribute of the at least one drug mᵢ is represented as X^{v}∈j^{N×L}^{ᵥ}, Lᵥ represents a dimension of the features of the v-th attribute, v=1, 2, . . . , V and V represents a number of the attributes;
calculating a temporal correlation between the one or more symptoms, the one or more drugs, and the one or more adverse reactions at different time points by:
calculating a probability p(sᵢ|sᵢ₋₁, sᵢ₋₂, . . . , s₀) of the at least one symptom sᵢ occurring at the time tᵢ using the following formula:
$$p ( {s}_{i} \vert {s}_{i - 1} , {s}_{i - 2} , … , {s}_{0} ) = \frac{p ( {s}_{i} , {s}_{i - 1} , {s}_{i - 2} , … , {s}_{0} )}{p ( {s}_{i - 1} \vert {s}_{i - 2} , … , {s}_{0} ) p ( {s}_{i - 2} , … , {s}_{0} )}$$
wherein p(sᵢ|sᵢ₋₁, sᵢ₋₂, . . . , s₀) represents a probability of an occurrence of the at least one symptom sᵢ at the time tᵢ, given a first set or subset of the one or more symptoms <s₀, s₁, . . . , sᵢ₋₁> occurring at a first set or subset of times t₀˜tᵢ₋₁, and p(sᵢ, sᵢ₋₁, sᵢ₋₂, . . . , s₀) represents a probability of a second set or subset of the one or more symptoms <s₀, sᵢ, . . . , sᵢ> occurring at a second set or subset of times t₀˜tᵢ;
calculating a probability of the at least one adverse reaction rᵢ occurring at the time tᵢ, p(rᵢ|sᵢ, mᵢ) using the following formula:
$$p ( {r}_{i} \vert {s}_{i} , {m}_{i} ) = p ( {s}_{i} \vert {s}_{i - 1} , {s}_{i - 2} , … , {s}_{0} ) \times p ( {m}_{i} \vert {s}_{i} ) \times h ( {s}_{i} , {m}_{i} ) \times {\prod }_{j = 0}^{i - 1} {w}_{i j} [ p ( {r}_{j} \vert {s}_{j} , {m}_{j} ) p ( {m}_{j} , {s}_{j} ) ]$$
where p(mᵢ|sᵢ) represents a probability of the at least one drug m being taken or administered based on the at least one symptom sᵢ, h(sᵢ, mᵢ) represents a first mapping relationship between the at least one symptom sᵢ, the at least one drug mᵢ, and the at least one adverse reaction rᵢ at a certain time, p(rⱼ|sⱼ, mⱼ) represents a probability of the one or more adverse reactions occurring based on at least one prior symptom sⱼ of the one or more symptoms and at least one prior drug mⱼ of the one or more drugs being taken or administered at a prior time tⱼ, and p(mⱼ, sⱼ) represents a joint probability of the at least one prior symptom sᵢ and the at least one prior drug mⱼ at the prior time tⱼ, and wᵢⱼ represents a weight of the at least one prior symptom sⱼ and the at least one prior drug mⱼ at the prior time tⱼ on the at least one adverse reaction rᵢ;
establishing a relationship between the one or more symptoms, the multi-attribute data of the one or more drugs, and the one or more adverse reactions by:
constructing a first similarity matrix Eₘ_{ᵢ}^{v} for the v-th attribute of the at least one drug mᵢ by computing a similarity between the features of the v-th attribute;
obtaining information about potential targets related to the symptoms and constructing a second similarity matrix Eₛ_{ᵢ} for the at least one symptom sᵢ based on a similarity between features of the potential targets;
establishing a first interaction relationship Uₘ_{ᵢ}_{, s}_{ᵢ}^{v} from the first similarity matrix Eₘ_{ᵢ}^{v} and the second similarity matrix Eₛ_{ᵢ} as follows:
$${U}_{{s}_{i} , {m}_{i}}^{v} = {\sum }_{a , b} {( {E}_{{m}_{i}}^{v} )}_{{a}^{.}}^{T} {( {E}_{{s}_{i}} )}_{{b}^{.}}$$
where (Eₘ_{ᵢ}^{v})_{a·}^{T} denotes a transpose of an a th row of the first similarity matrix Eₘ_{ᵢ}^{v}; (Eₛ_{ᵢ})_{b·} represents a b th row of the matrix Eₛ_{ᵢ}, and Uₘ_{ᵢ}_{, s}_{ᵢ}^{v} is an interaction and/or relationship between the v th attribute of the at least one drug mᵢ and the features of the potential targets of the at least one symptom sᵢ;
establishing a second interaction relationship Kₘ_{ᵢ}_{, s}_{ᵢ} between the attributes of the at least one drug mᵢ and the at least one symptom sᵢ according to:
$${K}_{{m}_{i} , {s}_{i}} = {\sum }_{v} {U}_{{m}_{i} , {s}_{i}}^{v}$$
constructing an objective function mapping of the one or more symptoms and the attributes of the one or more drugs to the one or more adverse reactions according to:
$$min {\sum }_{i = 0}^{n} {‖ {r}_{i} - f ( {K}_{{m}_{i} , {s}_{i}} ) ‖}_{2}^{2} + Ω ( f )$$
where ƒ represents a mapping function between the one or more drugs, the one or more symptoms and the one or more adverse reactions, Ω(ƒ) represents a regularization function for implicit variables in the function ƒ, the mapping function ƒ integrates a second mapping relationship between the interaction relationship Kₘ_{ᵢ}_{, s}_{ᵢ} of the at least one drug mᵢ and the at least one symptom sᵢ and the one or more adverse reactions at the time points or the time stamps; and
optimizing the mapping function ƒ to update one or more model parameters utilizing a stochastic gradient descent method;
constructing a drug adverse reaction prediction model as follows:
$$p ( {r}_{i} \vert {s}_{i} , {m}_{i} ) = p ( {s}_{i} \vert {s}_{i - 1} , {s}_{i - 2} , … , {s}_{0} ) \times p ( {m}_{i} \vert {s}_{i} ) \times f ( {K}_{{m}_{i} , {s}_{i}} ) \times {\prod }_{j = 0}^{i - 1} {w}_{i j} [ p ( {r}_{j} \vert {s}_{j} , {m}_{j} ) p ( {m}_{j} , {s}_{j} ) ]$$
where p(rᵢ|sᵢ, mᵢ) is a probability of the one or more patients having the at least one adverse reaction rᵢ at the time tᵢ; and
predicting the adverse drug reaction of the one or more patients at a next time point based on the drug adverse reaction prediction model.

2. The method as claimed in claim 1, wherein calculating the probability p(rᵢ|sᵢ, mᵢ) of the at least one adverse reaction rᵢ at the time tᵢ further includes:
disregarding the time points distant from the time tᵢ on the one or more adverse reactions at the time tᵢ, and considering only a temporal correlation tᵢ−tⱼ≤τ, whereby the at least one adverse reaction rᵢ at the first certain time tᵢ is influenced by the one or more symptoms, the one or more drugs, and the one or more adverse reactions at times t_{i-τ}˜tᵢ₋₁ as follows:
$$p ( {r}_{i} \vert {s}_{i} , {m}_{i} ) = p ( {s}_{i} \vert {s}_{i - 1} , {s}_{i - 2} , … , {s}_{i - τ} ) \times p ( {m}_{i} \vert {s}_{i} ) \times h ( {s}_{i} , {m}_{i} ) \times {\prod }_{j = i - τ}^{i - 1} {w}_{i j} [ p ( {r}_{j} \vert {s}_{j} , {m}_{j} ) p ( {m}_{j} , {s}_{j} ) ]$$
$$or$$
$$p ( {r}_{i} \vert {s}_{i} , {m}_{i} ) = p ( {s}_{i} \vert {s}_{i - 1} , {s}_{i - 2} , … , {s}_{i - τ} ) \times p ( {m}_{i} \vert {s}_{i} ) \times f ( {K}_{{s}_{i} , {m}_{i}} ) \times {\prod }_{j = i - τ}^{i - 1} {w}_{ij} [ p ( {r}_{j} \vert {s}_{j} , {m}_{j} ) p ( {m}_{j} , {s}_{j} ) ]$$
and a probability p(r₀|s₀, m₀) of an initial drug adverse reaction or set of drug adverse reactions r₀ at an initial time t₀ is:
$$p ( {r}_{0} \vert {s}_{0} , {m}_{0} ) = p ( {s}_{0} ) \times p ( {m}_{0} \vert {s}_{0} ) \times h ( {s}_{0} , {m}_{0} ) .$$

3. The method as claimed in claim 1, wherein the one or more specific diseases includes COVID-19, the one or more symptoms includes fever, sore throat, cough with sputum, runny nose, nausea and/or vomiting, the one or more drugs includes acetaminophen, hydroxychloroquine, bromhexine, chlorpheniramine, and/or promethazine, and the one or more adverse reactions includes indigestion, rash, hepatic and/or renal impairment, fatigue, dizziness, gastric pain, diarrhea, constipation, drowsiness, itching, headache, tremor, dry mouth, blurred vision and/or arrhythmia.

4. The method as claimed in claim 1, wherein the one or more specific diseases includes diabetes, the one or more symptoms includes thirst, polyuria, frequent urination, dizziness, blurred vision, shortness of breath, chest pain, palpitations, fatigue, sweating, seizures, blindness and/or metabolic disorders, the one or more drugs includes metformin, glipizide, insulin and/or captopril, and the one or more adverse reactions includes diarrhea, indigestion, abdominal pain, vomiting, dizziness, chills, nausea, sweating, anxiety, tachycardia, tremors or shivering, and/or palpitations.

5. The method as claimed in claim 1, wherein the one or more specific diseases includes hypertension, the one or more symptoms includes dizziness, headache, chest pain, neck discomfort, swelling of limbs, fatigue, blurred vision, numbness in limbs, palpitations and/or chest tightness, the one or more drugs includes enalapril, hydrochlorothiazide, amlodipine, losartan and/or propranolol, and the one or more adverse reactions includes fatigue, cough, indigestion, dry mouth, muscle spasms, nausea, edema, palpitations, muscle pain, atrioventricular blockage, drowsiness, dizziness and/or insomnia.

6. The method as claimed in claim 1, wherein the one or more specific diseases includes coronary heart disease, the one or more symptoms includes chest pain, back pain, chest tightness, tachycardia, shortness of breath, angina, and/or arm pain; the one or more drugs includes nitroglycerin, aspirin, bisoprolol and/or verapamil; and the one or more adverse reactions includes blurred vision, dry mouth, hypotension, nausea, vomiting, upper abdominal discomfort, gastrointestinal discomfort, fatigue, sweating, dizziness, constipation and/or palpitations.

7. The method as claimed in claim 1, wherein the one or more specific diseases includes chronic kidney failure, the one or more symptoms includes polyuria, cardiac arrhythmia, anorexia, oliguria, fatigue, vomiting, anorexia, sluggishness, gastrointestinal ulcers, and/or hematochezia; the one or more drugs includes benazepril, perindopril, hydrochlorothiazide, and/or furosemide; and the one or more adverse reactions includes headache, dizziness, nausea, cough, dry mouth, muscle spasms, fatigue, thirst, muscle soreness and/or arrhythmia.

8. The method of claim 1, further comprising determining that the one or more patients will have at least one of the one or more drug adverse reactions at the next time point when the probability p(rᵢ|sᵢ, mᵢ) is equal to or greater than a predetermined threshold.

9. The method of claim 8, wherein the predetermined threshold is 0.5 or higher.

10. The method of claim 8, further comprising determining the predetermined threshold based on a potential or likely severity of the one or more drug adverse reactions at the next time point.

11. The method of claim 10, wherein the predetermined threshold is from 0.1 to 0.5.

12. The method of claim 8, wherein the predetermined threshold is independent for each of the one or more drugs or each of the one or more adverse reactions.

13. A method of preventing one or more adverse reactions to one or more drugs, comprising:
predicting the one or more adverse drug reactions at the next time point according to the method of claim 1, and
withholding at least one of the one or more drugs from the one or more patients when the probability p(rᵢ|sᵢ, mᵢ) is equal to or greater than a predetermined threshold.

14. A method of treating one or more adverse reactions to one or more drugs, comprising:
administering the one or more drugs to one or more patients after the one or more patients present or exhibit one or more symptoms of one or more specific diseases,
predicting the one or more adverse drug reactions at a future time point according to the method of claim 1, and
prior to the one or more patients having or exhibiting the one or more adverse drug reactions at the future time point, either:
withholding the one or more drugs from the one or more patients, or
treating the predicted adverse drug reaction(s) in the one or more patients.

15. The method of claim 14, comprising treating the predicted adverse drug reaction(s) in the one or more patients.
